(19) United States
(12) Patent Application Publication
(10) Pub. No.: US 2008/0311050 A1
(43) Pub. Date: Dec. 18, 2008

(54) Method for Producing Shape Memory Effects in Hair in Connection with Hydrophobic Active Ingredients

(75) Inventors: Andreas Lendlein, Berlin (DE); Tareq Ali Abdulla Hasson, Aachen (DE); Karl Kratz, Berlin (DE); Leonid Kuper, Aachen (DE); Thomas Krause, Darmstadt (DE); Axel Kalbfleisch, Darmstadt (DE); Anja Duchscherer, Hadamar (DE); Angelika Beyer, Waldaschaff (DE)

Correspondence Address: SEED INTELLECTUAL PROPERTY LAW GROUP PLLC, 701 FIFTH AVE, SUITE 5400, SEATTLE, WA, 98104, US

(73) Assignee: MNEMOSCIENCE GMBH, UEBACH-PALENBERG (DE)

(21) Appl. No.: 10/566,230

(22) PCT Filed: Jul. 14, 2004
(86) PCT No.: PCT/EP2004/007800

§ 371 Date: Aug. 26, 2008

(30) Foreign Application Priority Data
Jul. 30, 2003 (DE) 103 34 788.7

(51) Int. Cl. A61K 8/06; A61K 9/107; A61Q 5/04

(52) U.S. Cl. 424/45; 424/70.2; 424/484

(57) ABSTRACT

A method for achieving a retrievable hair restyling is described. Here a composition is applied to the hair, said composition containing at least one first active ingredient or active ingredient complex which can provide a shape memory effect by itself or in combination with other substances after being applied to the hair and after the performance of the treatment of the hair according to the invention. The composition additionally contains at least one second active ingredient, namely a hydrophobic substance with a water solubility of ≦5 percent by weight at 20° C. The hair is arranged into a given (permanent) shape and said permanent shape is then fixed by inducing a chemical or physical modification of the applied active ingredients, wherein after a wanted or unwanted deformation of the shape memory, the original shape memory can be restored by means of a physical stimulation. In addition to the permanent hairstyle, a second (temporary) shape can be impressed. A method is also described for restoring a previously programmed permanent hairstyle that was temporarily transformed into a temporary shape or that was otherwise deformed.

The subject matter of the invention under consideration is a method for producing a retrievable hair restyling by using combinations of hydrophobic active ingredients and active ingredients that give the hair a shape memory effect, in particular, combinations of shape memory polymers or of macromers that can be cross-linked into shape memory polymers.

In the shaping of hair, a distinction is generally made between temporary and lasting, permanent hair styling. As a rule, a temporary hair styling takes place with the use of compositions based on solutions or dispersions of hair-fixing polymers. Products of this kind give the hair more or less hold, volume, elasticity, springiness and lustre by means of the addition of polymers. For example, in the form of a gel, these styling products facilitate the shaping and creation of a hairstyle; in the form of a hairspray, they improve the condition of a created hairstyle; and, in the form of fixing foams, they increase the volume of the hair. Detrimental is that the desired effects last for only a relatively short time and are quickly lost again as the result of outside influences, such as combing, wind, high air humidity or contact with water. As a rule, a permanent hair styling is accomplished by means of a permanent wave treatment. In this case, disulfide bonds in the hair are reductively cleaved, the hair is brought into a new shape and this new shape is fixed by forming new disulfide bonds by means of an oxidative process. Detrimental is that, as a result of the necessary chemical treatment of the hair with reductive and oxidation agents, it is not possible to avoid damage to the hair structure. A further disadvantage of the methods for hair restyling known up to now is that it is not possible to make the restyling reversible in a relatively simple manner, i.e., to move from one hairstyle to another without complex re-creation.

From JP 04-41416, hair cosmetics are known which contain specific linear polyurethanes having a glass transition temperature T_{g} of from 40-90° C. The method for hair treatment described therein corresponds to a treatment using typical thermoplastic materials. After the application of the composition, a hairstyle is prepared at a temperature above T_{g} and then fixed in place by cooling to below T_{g}. During a subsequent re-heating to a temperature above T_{g} the polymer softens, and a new hairstyle can be prepared. A method for a recallable, reversible hair restyling is not described.

The object underlying the present invention is to provide compositions having improved efficiency and performance with regard to providing a retrievable hair restyling with a high degree of recovery for a programmed hairstyle. Improved efficiency or performance can, for example, be improved adhesion to the hair, improved permanence of the effect, a higher degree of recovery of a programmed hairstyle, etc. A further object is to provide a method enabling a permanent hair restyling without damaging intervention in the hair structure. A further object is the provision of a method which enables the reversion of temporary styling shapes multiple times in a simple manner, and which makes it possible to revert to a previously prepared, programmed permanent hairstyle with a high degree of accuracy. A further object is the provision of a method which facilitates, in a simple manner and with a high degree of accuracy, reverse deformations of a hairstyle that have been introduced by external influences and to revert to a previously prepared, programmed permanent hairstyle.

This object is solved by a method for hair treatment, wherein
an active ingredient composition is applied to the hair, wherein the active ingredient composition

(A) contains at least a first active ingredient or first active ingredient complex, which are selected from or formed of substances, which, alone or in combination with other substances, can provide the hair with a shape memory effect after being applied to the hair and after the performance of the treatment described in the following, and

(B) contains at least a second active ingredient, which is selected from the hydrophobic substances;
the hair is arranged in a given shape (permanent memory shape) before, simultaneously with or after the application of the active ingredient composition and
the memory shape is subsequently fixed by inducing a chemical or physical modification of the applied active ingredients;

wherein after a wanted or unwanted deformation of the memory shape, the original memory shape can essentially be restored by means of physical stimulation.

Hydrophobic substances in the context of the invention are substances with a water solubility of ≦5 percent by weight, preferably ≦2 percent by weight, at 20° C. The weight ratio of (A):(B) can, for example, be from 1:100 to 1,000:1, from 1:50 to 200:1, from 1:10 to 5:1, or from 1:1 to 2.5:1.

One embodiment relates to a method of hair treatment using an active ingredient composition containing at least two substances, said substances having no or only weak shape memory properties individually and said substances giving a synergistically increased shape memory effect to the hair when applied together by means of the method according to the invention. In this connection, the at least two substances can comprise either the above-mentioned first active ingredient and the above-mentioned hydrophobic second active ingredient, or the at least two substances can form the above-mentioned active ingredient complex.

A special embodiment relates to a method for hair treatment, wherein
the first active ingredient of the composition applied to the hair is a cross-linkable macromer, which forms a shape memory polymer after cross-linking, wherein the macromer

a) contains cross-linkable regions that are cross-linkable through chemical bonds and

b) contains thermoplastic regions that are not chemically cross-linkable, and
the hair is arranged in a given (permanent) shape before, at the same time or afterwards and
the shape is subsequently fixed by chemically cross-linking the macromer, while forming the shape memory polymer,

wherein the shape memory polymer has at least one transition temperature Tₜᵣₐₙₛ.

A further object of the invention is a method for impressing a second hairstyle onto a first, programmed, retrievable hairstyle. In this connection, first a hairstyle (permanent shape) programmed by the method mentioned above is heated to a temperature above Tₜᵣₐₙₛ. The hair is then brought into the desired second (temporary) shape and the second shape is fixed by cooling to a temperature below Tₜᵣₐₙₛ.

A further object of the invention is a method for recovery of a first hairstyle (permanent shape) previously programmed by the above-mentioned method. For this purpose, a hairstyle in a temporary shape, or a hairstyle deformed by cold forming, is heated to a temperature above Tₜᵣₐₙₛ.

Shape memory polymers in the context of the invention are polymers from which materials can be produced that have the property that they allow any shape (permanent shape) to be impressed upon them, to which shape they recover, spontaneously and without the application of external forces, after a deformation or after the impression of a second shape (temporary shape), simply as a result of reheating or another energetic stimulus. At the same time, deformation and recovery are possible multiple times. The degree to which the original, permanent shape is achieved is, as a rule, somewhat less in a first relaxation cycle, consisting of deformation and recovery, than in subsequent cycles, presumably because of the elimination of fault spots, textures, etc., still existing at the beginning. An especially high degree of recovery is then achieved, however, in the subsequent relaxation cycles. The degree of recovery in the first relaxation cycle is preferably at least 30%, more preferably at least 40%, and, in the subsequent relaxation cycles, preferably at least 40%, particularly preferably at least 50%. The degree may, however, also be 75%, 90% or more. The degree of recovery can be measured in accordance with usual curl retention measurements by simply determining the length of a treated hair curl or by means of known, suitable stress-strain experiments. The shape memory effect on hair is the property wherein a specific hairstyle (permanent memory shape) may be substantially recalled after a deformation, spontaneously and without the application of any external forces, by simple heating or some other energetic stimulus, i.e., to a degree during the first relaxation cycle of at least 30%, particularly preferably at least 40%, and to a degree during subsequent relaxation cycles of preferably at least 40%, more preferably at least 50% or 75%.

In the context of the invention, macromers or pre-polymers which can be cross-linked into shape memory polymers are polymers or oligomers wherein the fixation of an impressed, permanent shape occurs by means of chemical bonds connecting individual polymer strands or oligomer strands. The cross-linkage by means of chemical bonds can be provided by means of ionic or covalent bonds. The cross-linking reaction can be any suitable chemical reaction, for example, a salt formation reaction, a condensation reaction, an addition reaction, a substitution reaction or a reaction initiated photochemically or by means of a radical. The cross-linking reaction can take place by using suitable catalysts or initiators, or it can take place without the use of a catalyst. The cross-linking reaction can be initiated by means of a suitable energy source, for example, electromagnetic radiation, ultrasound, heat or mechanical energy. Where necessary, a combination of two or more methods for initiation can be employed in order to increase the efficiency or the speed of the cross-linking reaction.

Shape-memory polymers which are suitable in accordance with the invention possess at least one transition temperature Tₜᵣₐₙₛ. This transition temperature can be a melting temperature Tₘ or a glass transition temperature T_{g}. Above Tₜᵣₐₙₛ, the polymer has a lower modulus of elasticity than below Tₜᵣₐₙₛ. The ratio of the moduli of elasticity below and above Tₜᵣₐₙₛ is preferably at least 20. The transition temperature Tₜᵣₐₙₛ is preferably above room temperature (20° C.), particularly at least 30° C., particularly preferably at least 40° C., and is the temperature above which the spontaneous recovery of the permanent shape occurs, starting from the deformed shape or from the temporary shape.

The terms “hairstyle” and “shape of hair” in the context of the invention are to be understood broadly and comprise, for example, also the degree of waviness or the degree of straightness of the hair. In the context of the invention, a programmed hairstyle is a group of hairs showing a specific shape due to shape memory polymers which have been cross-linked and fixed in a permanent shape. In the context of the invention, recovery of a programmed hairstyle means that the programmed hairstyle is recovered after a deformation, preferably to a degree of at least 40%, particularly preferably to a degree of at least 50% or 60%, in relation to the shape that arises after a first relaxation cycle. The degree of recovery can, for example, be determined by means of measuring the length of a hair curl or hair strand.

Suitable macromers or prepolymers that can be chemically cross-linked into shape memory polymers are macromonomers which can be polymerized or which can be cross-linked by means of single chemical bonds. The chemically cross-linked polymers are also designated as thermoset polymers in WO 99/42147. The macromers and thermoset polymers described in WO 99/42147 are suitable for use in accordance with the invention and are a constituent of this application. Soft, thermoplastic segments (switching segments) having a transition temperature Tₜᵣₐₙₛ are cross-linked by means of chemical, preferably covalent bonds. Consequently, switching segments and network points are required, wherein the network points fix the permanent shape and the switching segments fix the temporary shape. The shape memory effect is based on the change of the elasticity when the temperature falls above or below Tₜᵣₐₙₛ. The ratio of the moduli of elasticity below and above Tₜᵣₐₙₛ is preferably at least 20. The larger this ratio is, the more expressed the shape memory effect is. Four types of thermoset polymers having shape memory properties can be distinguished: Network polymers, interpenetrating networks, semi-interpenetrating networks and mixed interpenetrating networks. Network polymers can be formed by means of the covalent connection of macromonomers, i.e., of oligomers or polymers having connectable, reactive terminal groups, preferably ethylenically-unsaturated radically or photochemically reactive terminal groups. The cross-linking reaction can, for example, be started by means of light-sensitive or temperature-sensitive initiators or by means of red-ox systems or combinations thereof, or the reaction can be started without the use of initiators, e.g., by using UV light, heat or mechanical energy. Interpenetrating networks are formed from at least two components, each of which is cross-linked to itself but not with the other component. Mixed interpenetrating networks are formed from at least two components, wherein one component is cross-linked by means of chemical bonds and another component is cross-linked by means of physical interactions. Semi-interpenetrating networks are formed from at least two components, one of which is chemically cross-linkable, while the other one is not chemically cross-linkable, and both components cannot be separated by means of physical methods.

In principle, suitable are all synthetic or natural oligomers or polymers having reactive terminal groups or side groups which provide the cross-linked shape memory polymer with a suitable transition temperature Tₜᵣₐₙₛ and suitable moduli of elasticity above and below Tₜᵣₐₙₛ, wherein the terminal groups or the side groups are already present in a reactive form either during the manufacture or subsequently as a result of a derivatization, which oligomers or polymers allow a cross-linking reaction with the above-mentioned methods. Suitable macromers are, for example, macromers having the following formula

A1-(X)ₙ-A2 (I)

wherein A1 and A2 designate reactive, chemically cross-linkable groups and —(X)ₙ— designates a divalent, thermoplastic polymer or oligomer segment. A1 and A2 are preferably acrylate or methacrylate groups. The segment (X)ₙ preferably designates a polyester segment, an oligoester segment, a polyalkylene glycol segment, an oligoalkylene glycol segment, a polyalkylene carbonate segment or an oligoalkylene carbonate segment, wherein the alkylene groups are preferably ethylene groups or propylene groups. Suitable macromonomers for the formation of thermoset polymers having shape memory properties are oligo-(ε.-caprolactones) or poly(ε-caprolactones), oligolactides or polylactides, oligoalkylene glycols or polyalkylene glycols, e.g., polyethylene glycol or polypropylene glycol or their block copolymers, wherein the mentioned polymers or oligomers are substituted at a terminal position or side position by at least two ethylenically unsaturated groups, which can be polymerized by means of a radical reaction, e.g., acrylates or methacrylates.

The polymer segments can be segments derived from natural polymers, such as, for example, proteins or polysaccharides. They can also be synthetic polymer blocks. Suitable natural polymer segments are proteins, such as zein, modified zein, casein, gelatine, gluten, serum albumin or collagen, as well as polysaccharides such as alginates, celluloses, dextran, pullulan or polyhyaluronic acid, as well as chitin, poly(3-hydroxyalkanoate), particularly poly(β-hydroxybutyrate), poly(3-hydroxyoctanoates) or poly(3-hydroxy fatty acids). Also suitable are derivatives of natural polymer segments such as alkylated, hydroxyalkylated, hydroxylated or oxidized modifications. Synthetically modified natural polymers are, for example, cellulose derivatives, such as alkylcelluloses, hydroxyalkylcelluloses, cellulose ethers, cellulose esters, nitrocelluloses, chitosan or chitosan derivatives, which are, e.g., obtained by means of N-alkyl substitution and/or O-alkyl substitution or hydroxyalkyl substitution. Examples are methylcellulose, ethylcellulose, hydroxypropylcellulose, hydroxypropylmethylcellulose, hydroxybutylmethylcellulose, cellulose acetate, cellulose propionate, cellulose acetate butyrate, cellulose acetate phthalate, carboxymethylcellulose, cellulose triacetate or cellulose sulfate sodium salt. These materials are collectively designated in the following as “celluloses”.

Suitable synthetic polymer blocks include polyphosphazenes, poly(vinyl alcohols), polyamides, polyesteramides, polyamino-acids, polyanhydrides, polycarbonates, poly(lactide-co-glycolides), polyacrylates, polyalkylenes, polyacrylamides, polyalkylene glycols, polyalkylene oxides, polyalkylene terephthalates, polyorthoesters, polyvinylethers, polyvinylesters, polyvinylhalogenides, polyvinylpyrrolidones, polyesters, polylactides, polyglycolides, polysiloxanes, polyurethanes and copolymers thereof. Examples of suitable polyacrylates are poly(methylmethacrylate), poly(ethyl methacrylate), poly(butyl methacrylate), poly(isobutyl methacrylate), poly(hexyl methacrylate), poly(isodecyl methacrylate), poly(lauryl methacrylate), poly(phenyl methacrylate), poly(methyl acrylate), poly(isopropyl acrylate), poly(isobutyl acrylate) or poly(octadecyl acrylate). Suitable synthetic, easily biologically degradable polymer segments are polyhydroxy acids such as polylactides, polyglycolides and copolymers thereof, poly(ethylene terephthalate); poly(hydroxybutyric acid); poly(hydroxyvaleric acid); poly[lactide-co-(ε-caprolactone)]; poly[glycolide-co-(ε-caprolactone)]; polycarbonates, poly(amino acids); poly(hydroxyalkanoates); polyanhydrides; poly(orthoesters) as well as mixtures and copolymers thereof. Examples of polymer segments which are poorly biologically degradable are poly(methacrylic acid), poly(acrylic acid), polyamides, polyethylene, polypropylene, polystyrene, polyvinyl chloride, polyvinylphenol as well as mixtures and copolymers thereof.

In a particularly preferred embodiment, the composition comprises a mixture of (A1) macromers which are substituted with at least two reactive, cross-linkable groups, and (A2) macromers, which are substituted with only one reactive group. Suitable additional macromers are, for example, macromers of the following general formula:

R—(X′)ₙ-A3 (II)

wherein R designates a monovalent organic residue, A3 designates a reactive, chemically cross-linkable group and —(X′)n- designates a divalent, thermoplastic polymer segment or oligomer segment. A3 is preferably an acrylate group or a methacrylate group. The segment (X′)n preferably represents polyalkylene glycols, their monoalkyl ethers or their block copolymers, wherein the alkylene groups are preferably ethylene groups or propylene groups and wherein the alkyl groups preferably have from 1 to 30 C atoms. Particularly preferred are mixtures of (A1) polyalkylene glycols or polycaprolactones esterified at both terminal ends with acrylic acid or methacrylic acid and (A2) polyalkylene glycol monoalkyl ethers, esterified on one terminal end with acrylic acid or methacrylic acid, wherein the alkylene groups are preferably ethylene groups or propylene groups and the alkyl groups are preferably C1 to C30 alkyl groups. Preferred as component (A1), which can also be used alone, are: poly(ε-caprolactone)-dimethacrylate, poly(DL-lactide)-dimethacrylate, poly(L-lactide-co-glycolide)-dimethacrylate, poly(ethylene glycol)dimethacrylate, poly(propylene glycol)dimethacrylate, PEG-block-PPG-block-PEG-dimethacrylate, poly(ethylene adipate)-dimethacrylate, hexamethylene carbonate dimethacrylate. Suitable as component (B) are, e.g., the following: poly(ethylene glycol)monoacrylate, poly(propylene glycol)monoacrylate and monoalkyl ethers thereof.

A further particular embodiment relates to a method of treating hair, wherein
the first active ingredient of the composition applied to the hair is a shape memory polymer, which has at least two transition temperatures Tₜᵣₐₙₛ and T′ₜᵣₐₙₛ, and

a) has at least one hard segment with a first transition temperature T′ₜᵣₐₙₛ, which hard segment is cross-linkable by means of physical interaction, wherein the first transition temperature is above room temperature, preferably more than 10° C. above room temperature, and

b) has at least one soft segment having a second transition temperature Tₜᵣₐₙₛ, which lies below T′ₜᵣₐₙₛ, preferably at least 10° C. below T′ₜᵣₐₙₛ,
before, simultaneously or subsequently the hair is arranged into a defined (permanent) shape and
the shape is subsequently fixed by physical cross-linking of the shape memory polymers.

The shaping of the hair suitably occurs under heating to a temperature of at least T′ₜᵣₐₙₛ, and the shape of the hair is fixed by means of cooling to a temperature below Tₜᵣₐₙₛ. Room temperature usually refers to the ambient temperature, preferably at least 20° C. and, in warmer climates, preferably at least 25° C. The application of the composition onto the hair can be carried out in different ways, for example, directly by spraying or indirectly by applying the composition first to the hand or a suitable device, for example a comb, a brush etc., followed by a subsequent distribution in or onto the hair. The consistency of the composition can, for example, be that of a solution, dispersion, lotion, thickened lotion, gel, foam or a semi-solid compound, or it can be creamy or waxy.

A further object of the invention is a method for impressing a second hairstyle on to a programmed, retrievable first hairstyle. In this connection, first a hairstyle (permanent shape), programmed by means of the above-mentioned method, is heated to a temperature between T′ₜᵣₐₙₛ and Tₜᵣₐₙₛ. The hair is then brought into the desired second (temporary) shape and the second shape is fixed by cooling to a temperature below Tₜᵣₐₙₛ.

A further object of the invention is a method for recovering a first hairstyle (permanent shape), programmed by means of the above-mentioned method. For this purpose, a hairstyle in a temporary shape, or a hairstyle deformed by cold-forming, is heated to a temperature above Tₜᵣₐₙₛ. The permanent shape is formed again spontaneously and automatically. Cold-forming of a hairstyle means a change of the hairstyle at the ambient temperature, without the supply of additional heat by means of a hairdryer or similar devices. The deformation can, for example, be caused mechanically, for example, by means of simply hanging the curls under the influence of gravity, by means of combing or brushing the hair, as a result of the wind or humidity, as the result of mechanical influences during sleep or while lying down, etc.

The invention furthermore relates to a method for reprogramming a permanent hairstyle, previously programmed according to the above-mentioned method, into a different, new permanent shape. For this purpose, the original hairstyle is heated to a temperature above T′ₜᵣₐₙₛ and the hair is brought into a new shape. This new shape is subsequently fixed by means of cooling to a temperature below T′ₜᵣₐₙₛ.

In the context of the invention, physically cross-linkable shape memory polymers are polymers with which the fixation of the impressed permanent shape due to cross-linking occurs by physical interactions. A cross-linking by means of physical interactions can be achieved when specific segments of the polymer chains assemble together into crystalline areas. The physical interactions can be charge transfer complexes, hydrogen bonds, dipolar interactions or hydrophobic interactions, Van der Waals interactions or ionic interactions of polyelectrolyte segments. The interactions can occur between different segments within one polymer strand (intramolecular) and/or between different polymer strands (intermolecular). The formation of the interactions can, for example, be initiated by means of cooling (in particular, in cases of crystallisation) and/or by means of drying, i.e., the removal of solvents.

Suitable physically cross-linkable shape memory polymers according to the invention have at least two transition temperatures Tₜᵣₐₙₛ and T′ₜᵣₐₙₛ. The two transition temperatures can, for example, be melting temperatures Tₘ or glass transition temperatures T_{g}. Above Tₜᵣₐₙₛ, the polymer has a lower modulus of elasticity than it does below Tₜᵣₐₙₛ. The ratio of the moduli of elasticity below and above Tₜᵣₐₙₛ is preferably at least 10, particularly preferably at least 20. The lower transition temperature Tₜᵣₐₙₛ is preferably higher than room temperature (20° C.), in particular at least 30° C., particularly preferably at least 35° C. or at least 40° C., and is that temperature which, when exceeded, sees the spontaneous recovery of the permanent shape from the deformed or temporary shape. Tₜᵣₐₙₛ is preferably so far above the usually to be expected ambient temperatures that at the ambient temperature, no significant, unintentional, thermally induced shaping of the temporary hairstyle occurs. Suitable ranges for Tₜᵣₐₙₛ are, for example, from 25 to 100° C., from 30 to 75° C., from 35 to 70° C. or from 40 to 60° C. The upper transition temperature T′ₜᵣₐₙₛ is higher than Tₜᵣₐₙₛ and is that temperature above which the impression of the permanent shape or the re-impression of a permanent shape into a new permanent shape occurs, and below which the permanent shape is fixed. T′ₜᵣₐₙₛ is preferably so much higher than Tₜᵣₐₙₛ that during the heating of the hairstyle to a temperature above Tₜᵣₐₙₛ for the recovery of the permanent hairstyle or for the reforming of a temporary hairstyle while maintaining the permanent hairstyle, no significant, unintentional, thermally induced deformation of the permanent shape occurs. Preferably, T′ₜᵣₐₙₛ is at least 10° C., particularly preferably at least 20° C. or at least 30° C. above Tₜᵣₐₙₛ. The difference between T′ₜᵣₐₙₛ and Tₜᵣₐₙₛ can, for example, be from 10 to 80° C., from 20 to 70° C. or from 30 to 60° C. Suitable ranges for T′ₜᵣₐₙₛ are, for example, from 40 to 150° C., from 50 to 100° C. or from 70 to 95° C.

Suitable physically cross-linked shape memory polymers are polymers which consist of at least one hard segment and at least one soft segment. The hard segment has physical cross-links and has a transition temperature T′ₜᵣₐₙₛ which is above room temperature, preferably more than 10° C. above 20° C. The soft segment has a transition temperature Tₜᵣₐₙₛ which is below T′ₜᵣₐₙₛ, preferably at least 10° C. below T′ₜᵣₐₙₛ. The polymer segments are preferably oligomers, in particular, linear chain molecules having a molecular weight of, for example, 400 to 30,000, preferably 1,000 to 20,000 or 1,500 to 15,000. These can be linear di-block, tri-block, tetra-block or multi-block copolymers; they can be branched, dendritic or grafted copolymers. Preferably, these polymers are not linear polyether urethanes containing bis(2-hydroxy-ethyl)-hydroquinone. The molecular weight of the polymers can, for example, be from 30,000 to 1,000,000, preferably from 50,000 to 700,000 or from 70,000 to 400,000. Suitable physically cross-linked shape memory polymers are described in WO 99/42147 and are referred to there as thermoplastic polymers. The thermoplastic polymers described in WO 99/42147, as well as the methods of production described there, are suitable according to the invention and are a constituent of this application. These have a degree of crystallinity of preferably from 3 to 80%, more preferably from 3 to 60%. The ratio of the moduli of elasticity below and above Tₜᵣₐₙₛ is preferably at least 10, particularly preferably at least 20. The polymer segments can be segments derived from natural polymers, such as proteins or polysaccharides, for example. They can also be synthetic polymer blocks. Suitable natural or synthetic polymer segments are identical to those named above for the cross-linkable macromers.

Suitable shape memory polymers are, in particular, multiblock copolymers, which have at least one first type of blocks and at least one second type of blocks, differing from the first type, wherein the blocks have the effect that the multiblock copolymer has two different transition temperatures. Suitable multiblock copolymers are, in particular, such copolymers as are produced from at least two different macrodiols and at least one diisocyanate. Macrodiols are oligomers or polymers having at least two free hydroxyl groups. Oligomers usually consist of at least two, preferably at least three, particularly 4 to 20, 5 to 15 or 6 to 10 monomers. The macrodiols may possess the general formula HO-A-OH, wherein A represents a divalent oligomeric or polymeric group, preferably a polyester or an oligoester. The diisocyanate can have the general formula OCN-B-NCO, wherein B represents a divalent organic group, preferably an alkylene group or an arylene group, which can be substituted with further substituents. The alkylene group can be linear, branched or cyclic, preferably having 1 to 30 C atoms, more preferably 2 to 20 or 5 to 15 C atoms.

Particularly preferred shape memory polymers are the copolyester urethanes described in WO 99/42147, in particular the reaction products of (a) two different macrodiols, selected from α, ω-dihydroxy polyesters, α, ω-dihydroxy oligoesters, α, ω-dihydroxy polylactones and α, ω-dihydroxy oligolactones, and (b) at least one diisocyanate, preferably trimethylhexane-1,6-diisocyanate. Particularly preferred are macrodiols from poly(para-dioxanone) (PDX), poly(pentadecalactone) (PDL), poly(ε-caprolactone) (PCL), poly(L-lactide-co-glydolide) (PLGA). The molar masses of the macrodiols are preferably in the range of from 400 to 30,000, preferably 1,000 to 20,000 or 1,500 to 15,000. The molar masses of the resulting multiblock copolymers are preferably M_{w}=30,000 to 1,000,000, particularly preferably from 50,000 to 700,000 or from 70,000 to 400,000 g/mol, which can be determined by GPC. The polydispersities are preferably within the range from 1.7 to 2.0

### Hydrophobic Active Ingredients

Suitable hydrophobic active ingredients are, for example, oils, fats, waxes, fatty alcohols, fatty acids, hydrophobic polymers, hydrophobic tensides, hydrophobic silicone compounds, etc. Suitable hydrophobic tensides are particularly those having HLB levels≦7, e.g., ethoxylated C8 to C30, preferably C10-C20, fatty alcohols with an ethoxylation degree of preferably 1, 2 or 3, e.g., Ceteareth-2, Ceteareth-3, Ceteth-1, Ceteth-2, Ceteth-3, Laureth-1, Laureth-2, Laureth-3, Myreth-2, Myreth-3, Oleth-2, Oleth-3, Steareth-2, Steareth-3. Suitable oils are, for example, those having a melting point of less than 25° C. and a boiling point of preferably more than 250° C., particularly more than 300° C. Vegetable or animal oils, mineral oils (Paraffinum Liquidum), oily fatty acid esters or fatty alcohol esters can be considered. Furthermore, hydrocarbon oils, e.g., paraffin oils or isoparaffin oils, squalane, oils from fatty acids and polyols, in particular triglycerides are also suitable. Suitable vegetable oils are, for example, sunflower oil, coconut oil, castor oil, lanolin oil, jojoba oil, corn oil, soy oil. Suitable waxes are, for example, animal, vegetable, mineral and synthetic waxes, microcrystalline waxes, macrocrystalline waxes, solid paraffins, ozocerite, mountain wax, Fischer-Tropsch waxes, polyolefin waxes, e.g., polybutylene, beeswax, wool wax and its derivatives, such as wool wax alcohols, candelilla wax, Carnauba wax, Japan wax, hydrogenated fats, fatty acid esters and fatty acid glycerides with a solidification point in each case of more than 40° C., wax alcohols, wax acids and polyethylene waxes, etc. The waxes or wax-like substances have a solidification point of more than 40° C., preferably more than 55° C. Suitable hydrophobic soft wax substances are, for example, Vaseline or semi-solid paraffins, e.g., petrolatum (semi-solid mixture of hydrocarbons extracted from petroleum). The solidification points are usually in the range of from approximately 25° C. to 40° C.

Hydrophobic polymers are preferably selected from polymers that contain at least one type of monomer, which is selected from acrylic acid alkyl esters and methacrylic acid alkyl esters, wherein the alkyl groups have at least 6 C atoms, preferably 8 to 30 C atoms.

Hydrophobic silicone compounds are preferably selected from silicone oils, silicone waxes, dimethylpolysiloxanes and dimethylsiloxane/alkylmethylsiloxane copolymers, wherein the alkyl groups have at least 6 C atoms, preferably 8 to 30 C atoms. Suitable silicone compounds are, e.g., silicone waxes, polydimethylsiloxane(dimethicone), phenylated silicones, polyphenylmethylsiloxanes, phenyltrimethicones, methylpolysiloxanes with alkyl side chains, wherein the alkyl groups have 2 to 30, preferably at least 8, C atoms, more preferably 10 to 20 C atoms (e.g., stearyl dimethicones), α-Hydro-ω-hydroxypolyoxydimethylsilylene(dimethiconol), cyclic dimethylpolysiloxane(cyclomethicone), trimethyl(octadecyloxy)silane(stearoxytrimethylsilane), dimethylsiloxane/glycol copolymer(dimethicone copolyol), dimethylsiloxane/aminoalkylsiloxane copolymer having terminal hydroxy groups (amodimethicone), monomethylpolysiloxane having lauryl side chains and terminal polyoxyethylene chains and/or polyoxypropylene chains (lauryl methicone copolyol), dimethylsiloxane/glycol copolymer acetate(dimethicone copolyol acetate), dimethylsiloxane/aminoalkylsiloxane copolymer having terminal trimethylsilyl groups (trimethylsilylamodimethicone). Preferred silicone polymers are dimethicones, cyclomethicones and dimethiconols. Mixtures of silicone polymers are also suitable, for example a mixture of dimethicone and dimethiconol. The above designations provided in brackets correspond to INCI (International Cosmetic Ingredients) nomenclature employed for the designation of cosmetic active ingredients and additives.

Particularly preferred hydrophobic substances are dimer acids and their derivatives. Dimer acids are dimerized fatty acids, particularly the dimers of simple, unsaturated C8 to C30 fatty acids, e.g., oleic acid or tall oil fatty acid (TOFA). Derivatives are, e.g., dimerdiol, dimerdiol oligoether and oligoester diol. Dimerdiol is the designation for α, ω-C36 diols, which can be produced by the dimerization of oleyl alcohol or by the hydrogenation of dimer fatty acids or their esters. Oligoester diols can be produced from dimerdiol by means of acid-catalyzed dehydration. Oligoester diols are the dihydroxyesters from dicarboxylic acids and diols with a fatty basis. The molecular weight of the dimerdiol oligoethers and the oligoester diols is preferably from 1,000 to 10,000, particularly preferably approximately 2,000 to 5,000. Preferred oligomers are particularly dimers, trimers and tetramers. Preferred, commercially available diols are dimer diols (Sovermol® 908, Cognis, molecular weight approximately 550 g/mol) and dimerdiol oligoethers (Sovermol® 909, molecular weight approximately 1,000 g/mol and Sovermol® 910, molecular weight approximately 2,000 g/mol).

The hydrophobic ingredients can be low-volatility or high-volatility substances, wherein low-volatility substances that remain on the hair are preferred, particularly those having an evaporation rate of preferably more than 20, particularly preferably more than 100. The evaporation rate is defined as the quotient of the evaporation time for the liquid to be tested and the evaporation time of diethylether as the comparison liquid. In a preferred embodiment, the hair treatment composition according to the invention contains both at least one low-volatility hydrophobic substance and at least one high-volatility hydrophobic substance with an evaporation rate of less than 20.

Compositions for the treatment of hair according to the invention comprise the first active ingredient, which alone or in combination with an additional substance is responsible for the shape memory effect, in an amount of preferably from 0.01 to 25 percent by weight, particularly preferably 0.1 to 15 percent by weight, in a suitable medium. Preferred amounts of the hydrophobic active ingredients are from 0.01 to 10 percent by weight, particularly preferably from 0.05 to 5 percent by weight. The composition may be present as a solution, dispersion, emulsion, suspension or latex. The liquid, gel-type, semi-solid or solid medium here is substantially cosmetically acceptable and physiologically harmless.

The composition according to the invention is generally present in the form of a solution or dispersion in a suitable solution agent or dispersion agent. Particularly preferred are water, organic solvents and mixtures of water and at least one organic solvent. Preferred organic solvents are, e.g., alcohols and acetone. Suitable solvents are, e.g., aliphatic linear or branched C1 to C4 alcohols or a mixture of water with one of these alcohols. Other organic solvents can, however, also be employed, wherein, in particular, unbranched or branched hydrocarbons, such as pentane, hexane, isopentane, cyclic hydrocarbons such as cyclopentane and cyclohexane, organic linear or cyclic ethers, such as tetrahydrofurane (THF) or liquid organic esters, such as ethylacetate, can be mentioned. Furthermore, solvents on the basis of silicone are also suitable, in particular silicone oils on the basis of linear or cyclic polydimethylsiloxanes (dimethicones or cyclomethicones), wherein volatile silicones with a boiling point of less than 200° C. are preferred. Additional solvents are acetone, tetrahydrofurane, chloroform, etc. The solvents are preferably present in an amount from 0.5 to 99 percent by weight or more preferably in an amount from 10 to 97 percent by weight, from 20 to 95 percent by weight or from 40 to 90 percent by weight.

The compositions according to the invention can additionally contain from 0.01 to 25 percent by weight of at least one hair care active ingredient, hair-fixative active ingredient and/or hair-colouring active ingredient. Hair fixatives are, in particular, the known conventional film-forming hair fixative polymers. The film-forming and hair-fixative polymer can be of synthetic origin or of natural origin, and can be of nonionic, cationic, anionic or amphoteric character. Such a polymer additive, which can be present in an amount of from 0.01 to 25 percent by weight, preferably 0.1 to 20 percent by weight, particularly preferably from 0.5 to 15 percent by weight, can also consist of a mixture of more than one polymer, and this additive can be modified further with respect to the hair-fixative properties by means of the addition of further polymers having a thickening effect. Film-forming, hair-fixative polymers according to the invention are polymers which are able, when employed in aqueous, alcoholic or aqueous-alcoholic solutions at a concentration of from 0.01 to 5%, to provide a polymer film on the hair, thereby fixing the hair in this manner.

Homopolymers of vinyl pyrrolidone, homopolymers of N-vinyl formamide, copolymerisates of vinyl pyrrolidone and vinyl acetate, terpolymers of vinyl pyrrolidone, vinyl acetate and vinyl propionate, polyacrylamides, polyvinyl alcohols or polyethylene glycols having a molecular weight of from 800 to 20,000 g/mol can be employed as suitable, synthetic, nonionic, film-forming, hair-fixative polymers in the hair treatment composition according to the invention. Suitable synthetic, anionic, film-forming polymers are crotonic acid/vinyl acetate copolymers and terpolymers of acrylic acid, ethyl acrylate and N-t-butylacrylamide. Natural film-forming polymers or polymers obtained from these by means of chemical conversion can also be employed in the hair treatment composition according to the invention, for example Chinese balsam resin, cellulose derivatives such as hydroxypropylcellulose having a molecular weight of from 30,000 to 50,000 g/mol, or shellac in a neutralized or unneutralized form. Amphoteric polymers can also be employed in the hair treatment composition according to the invention. Suitable are, e.g., copolymers of octylacrylamide, t-butylaminoethylmethacrylate and two or more monomers selected from the group consisting of acrylic acid, methacrylic acid and simple esters thereof.

The consistency of the hair treatment composition according to the invention can be increased by means of the addition of thickeners. In this respect, homopolymers of acrylic acid having a molecular weight of from 2,000,000 to 6,000,000 g/mol are suitable, for example. Copolymers of acrylic acid and acrylamide (sodium salt) having a molecular weight of from 2,000,000 to 6,000,000 g/mol, sclerotium gum and copolymers of acrylic acid and methacrylic acid are also suitable.

A cosmetic composition according to the invention can be used in various forms of application, for example, as a lotion, spray lotion, cream, gel, foam-gel, aerosol spray, non-aerosol spray, aerosol foam, non-aerosol foam, as an o/w emulsion or w/o-emulsion, as a micro emulsion or as a hair wax.

If the hair treatment composition according to the invention is provided in the form of an aerosol spray, the composition additionally contains from 15 to 85 percent by weight, preferably from 25 to 75 percent by weight, of a propellant and the composition is filled into a pressurized container having a spray head. Suitable as propellants are lower alkanes, such as n-butane, isobutene and propane, as well as mixtures thereof, as well as dimethyl ethers or fluorohydrocarbons, such as F 152a (1,1-difluoroethane) or F 134 (tetrafluoroethane), as well as propellants which are present at the pressures in question in gaseous forms such as N₂, N₂O and CO₂, as well as mixtures of the propellants mentioned in the preceding.

If the hair treatment composition according to the invention is provided in the form of a sprayable non-aerosol hairspray, the composition is sprayed by means of a suitable, mechanically operated spraying device. Spray devices of the mechanical type are devices which allow the spraying of a composition without using a propellant. Suitable mechanical spray devices are, e.g., spray pumps or elastic containers provided with a spray valve, into which the cosmetic composition according to the invention is filled under pressure, wherein the elastic container is expanded and wherein the cosmetic composition is continuously discharged if the valve is opened, due to the contraction of the elastic container.

If the hair treatment composition according to the invention is provided in the form of a hair foam (mousse), the composition contains at least one conventional foaming substance known in the art for this purpose. The composition is foamed with or without the aid of propellant gases or chemical propellants and worked into the hair as a foam, and then left in the hair without being rinsed out. A product according to the invention has, as an additional component, a device for foaming the composition. Considered as devices for foaming are devices which allow a liquid to be foamed with or without the use of a propellant. For example, a commercially available foam pump or an aerosol foaming head can be used as a suitable mechanical foaming device.

If the hair treatment composition according to the invention is provided in the form of a hair gel, it contains additionally at least one gel-forming substance in an amount of preferably from 0.05 to 10, more preferably from 0.1 to 2, percent by weight. The viscosity of the gel preferably amounts to from 100 to 50,000 mm²/s, particularly preferably from 1,000 to 15,000 mm²/s at 25° C., measured as dynamic viscosity using a Bohlin Rheometer CS, measurement body C25 using a shear velocity of 50 s⁻¹.

If the hair treatment composition according to the invention is provided in the form of a hair wax, it contains water-insoluble fatty substances or waxy substances, or substances in an amount that provide the composition with a waxy consistency, in an amount of preferably 0.5 to 30 percent by weight. Suitable water-insoluble substances are, for example, emulsifiers having an HLB-value of below 7, silicone oils, silicone waxes, wax materials (e.g., wax alcohols, wax acids, wax esters, as well as, in particular, natural waxes such as beeswax, Carnauba wax, etc.), fatty alcohols, fatty acids, fatty acid esters or high-molecular polyethylene glycols having a molecular weight of from 800 to 20,000, preferably from 2,000 to 10,000, g/mol.

If the hair treatment composition according to the invention is provided in the form of a hair lotion, it is present as a substantially non-viscous or low-viscosity solution, dispersion or emulsion, each capable of flowing, with a content of at least 10 percent by weight, preferably 20 to 95 percent by weight, of a cosmetically compatible alcohol. As alcohols, particularly the lower C1 to C4 alcohols usually used for cosmetic purposes can be used, such as ethanol and isopropanol, for example.

If the hair treatment composition according to the invention is present in the form of a hair cream, it is preferably provided in the form of an emulsion and either it contains additional viscosity-providing components in an amount of from 0.1 to 10 percent by weight or the required viscosity and creamy consistency are built up in the usual way by means of micelle formation with the help of suitable emulsifiers, fatty acids, fatty alcohols, waxes, etc.

In a preferred embodiment the composition according to the invention is capable of simultaneously facilitating both the impression of a retrievable hairstyle and a provision of hair coloration. The composition is then formulated as a colouring hair treatment composition, e.g., as a colouring fixative, a colouring cream, a colouring foam, etc. It then contains at least one colouring substance. This colouring substance component can be an organic colorant, in particular a so-called direct dye, or it can also be an inorganic pigment. In the substance according to the invention, the total amount of colouring substance amounts to about 0.01 to 7 percent by weight, preferably to about 0.2 to 4 percent by weight. Suitable direct dyes to be used in the composition according to the invention are, e.g., triphenylmethane colorants, aromatic nitro colorants, azo colorants, quinone colorants or cationic or anionic colorants.

Suitable hair-colouring pigments are practically insoluble colorants in the medium of the composition, and these pigments may be of organic or inorganic nature. Inorganic-organic mixed phase pigments are also possible. The pigments are preferably not nanopigments. The preferred particle size is from 1 to 200 μm, particularly 3 to 150 μm, particularly preferably 10 to 100 μm. Preferred are inorganic pigments.

The hair treatment composition according to the invention preferably additionally contains at least one hair-care substance in an amount of from 0.01 to 10, particularly preferably of from 0.05 to 5, percent by weight. Preferred hair-care substances are cation-active substances. Cation-active substances are characterized either in that they carry at least one permanent cationic group within the molecule, for example an iminium group or an ammonium group, in particular a quaternary ammonium group, or in that they carry at least one group which can be rendered cationic, for example a primary, secondary or tertiary amine group, which can be rendered cationic by protonation, wherein quaternary ammonium groups are preferred. The cation-active agent is a substance which possesses a substantivity to human hair due to the cationic group or the group which may be rendered cationic. Suitable cation-active substances are, for example, tensides having cationic groups or groups which can be rendered cationic, in particular cationic tensides, betainic or amphoteric tensides; polymers having cationic groups or groups which may be rendered cationic, in particular cationic, betainic or amphoteric polymers; silicone compounds having cationic groups or groups which may be rendered cationic, in particular diquaternary or polyquaternary siloxanes or amodimethicones; cationically derivatized proteins; cationically derivatized protein hydrolysates or betaine.

Usually additional known cosmetic additives can be added to the hair treatment composition according to the invention, for example non-fixative non-ionic polymers, such as polyethylene glycols, non-fixative, anionic and natural polymers, as well as mixtures thereof, in amounts of preferably from 0.01 to 50 percent by weight. It is also possible to add perfume oils and aromatic substances in an amount of from 0.01 to 5 percent by weight, opacifiers such as ethylene glycol distearate in an amount of from 0.01 to 5 percent by weight, wetting agents or emulsifiers, particularly anionic, or nonionic tensides, such as fatty alcohol sulphates, ethoxylated fatty alcohols, fatty acid alcanolamides, such as esters of hydrated fatty acids from castor oil, in an amount of from 0.1 to 30 percent by weight, as well as moisturizers, dye-receptivity improving agents, light protection agents, anti-oxidative agents and preservatives in amounts of from 0.01 to 10 percent by weight.

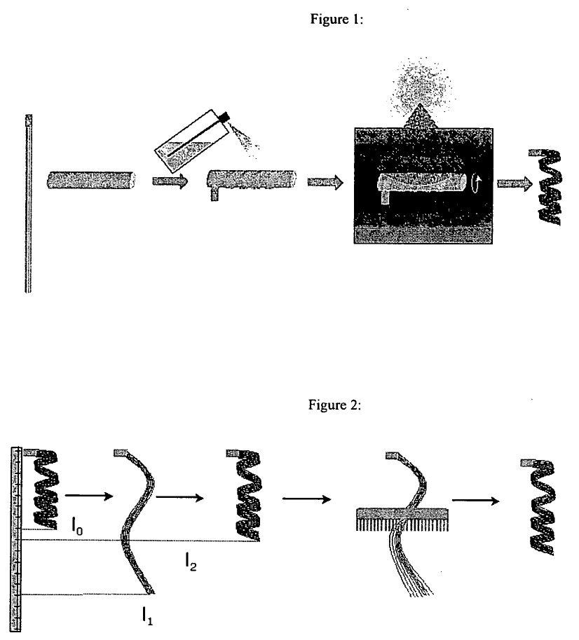

FIG. 1 schematically shows one possible method for producing a retrievable, permanent hairstyle. A strand of hair is wound onto a hair curler and sprayed with a solution according to the invention that contains a cross-linkable macromer. By irradiation with a suitable source of energy, for example, a UV lamp, the desired permanent shape is fixed. Subsequently the curler is removed.

FIG. 2 shows the deformation of a permanent hairstyle and the recovery of the permanent shape starting from the temporary shape. The hair curl in the permanent shape possesses a length l₀. The curl in the deformed shape has a length l₁. The curl in the recovered shape has a length l₂. The degree of recovery can be calculated as follows: Recovery=(l₁−l₂)/(l₁−l₀).

As a measure for the evaluation of the shape memory properties of a composition, the memory factor can be employed, which considers the formability of a permanent hairstyle into a temporary shape (shaping factor), as well as the restoration of the permanent shape, starting from the temporary shape (restoration factor, degree of recovery). If one starts with a straight strand of hair onto which a curly shape has been impressed as a permanent shape and onto which, subsequently, a second, straight shape has been impressed as a temporary shape, the shaping factor can be determined according to the following criteria:

| Degree of straightness | Shaping factor |
|---|---|
| Strongly wavy along the entire length | 0 |
| Slightly wavy from hairline to tip of hair | 1 |
| Hairline straight, tip with curl | 2 |
| Hairline straight, curvature at the tip | 3 |
| Straight all the way from the hairline to the tip | 4 |

The restoration factor can be determined according to the following criteria:

| Degree of restoration of the permanent shape | Restoration factor |
|---|---|
| 0% | 0 |
| 30% | 1 |
| 40% | 2 |
| 50% | 3 |
| 60% | 4 |
| 75% | 5 |
| 100% | 6 |

The memory factor M can be calculated with the following equation using the shaping factor f, the maximum shaping factor F=4, the restoration factor r and the maximum restoration factor R=6:

M=(f/F)*(r/R)*100

The memory factor should ideally be not below 25, preferably between 25 and 33.3, particularly preferably between 37 and 100.

The following examples are intended to illustrate the subject matter of the invention further.

### EXAMPLES

### Examples 1-12

Compositions according to Table 1 were prepared (amounts are given in grams).

TABLE 1

| | 1 | 2 | 3 | 4 | 5 | 6 |
|---|---|---|---|---|---|---|
| PEG-8k-DMA¹⁾ | 2.85 | 2.85 | 2.85 | 2.85 | 2.85 | 2.85 |
| Dimerdiol²⁾ | 0.15 | — | — | — | — | — |
| Dimerdiol oligoether³⁾ | — | 0.15 | — | — | — | — |
| Oligoester diol⁴⁾ | — | — | 0.15 | — | — | — |
| (Mₙ = 1000-3000) | | | | | | |
| Dimerdiol - DMA⁵⁾ | — | — | — | 0.15 | — | — |
| Dimerdiol oligoether- | — | — | — | — | 0.15 | — |
| DMA⁶⁾ | | | | | | |
| Oligoester diol-DMA⁷⁾ | — | — | — | — | — | 0.15 |
| (Mₙ = 1000-3000) | | | | | | |
| Ethanol/water/acetone | Ad 100 | Ad 100 | Ad 100 | Ad 100 | Ad 100 | Ad 100 |
| 45:45:10 | | | | | | |
| Memory factor | 44 | 45 | 43 | 45 | 44 | 43 |
| | 7 | 8 | 9 | 10 | 11 | 12 |
| PEG-8k-DMA¹⁾ | 2.85 | 2.85 | 2.85 | 2.85 | 2.85 | 2.7 |
| Dimer acid⁸⁾ | 0.15 | — | — | — | — | — |
| PMS-divinyl ether⁹⁾ | — | 0.15 | — | — | — | — |
| Castor oil | — | — | 0.15 | — | — | — |
| Ceraphyl ® RMT¹⁰⁾ | — | — | — | 0.15 | — | — |
| SilCare ® 41M65¹¹⁾ | — | — | — | — | 0.15 | — |
| Glycerin | — | — | — | — | — | 0.3 |
| monostearate | | | | | | |
| Ethanol/water/acetone | Ad 100 | Ad 100 | Ad 100 | Ad 100 | Ad 100 | Ad 100 |
| 45:45:10 | | | | | | |
| Memory factor | 40 | 35 | 35 | 37 | 40 | 38 |

¹⁾Polyethylene glycol with a molecular weight of 8,000, terminally esterified with methacrylic acid
²⁾α,ω-c36-Diol (Cognis)
³⁾Oligomer of Dimerdiol²⁾ (Cognis)
⁴⁾Synthetic oligoester diol of dicarboxylic acids and diols with a fatty basis
⁵⁾Like²⁾, terminally esterified with methacrylic acid
⁶⁾Like³⁾, terminally esterified with methacrylic acid
⁷⁾Like⁴⁾, terminally esterified with methacrylic acid
⁸⁾α,ω-C36-dicarboxylic acid (Cognis)
⁹⁾A product of the Aldrich Company
¹⁰⁾Castoryl maleate, a product of the ISP Company
¹¹⁾Stearyl dimethicone, a product of the Clariant Company

The memory factor M is determined according to the methods described above.

For the preparation of the permanent shape, a strand of hair having a length of 20 cm, having been moistened with water, was wound onto a curler and the polymer solution was applied thereon (20 to 30 mg polymer/g hair). The treated strand of hair was then fixed at 70° C. for 30 minutes with application of UV light. After cooling to room temperature (about 25° C.), the hair curler was removed. The curled strand (impressed permanent shape) had a length of about 4.5 cm. In order to produce a temporary shape (e.g., straight), the curled strand was heated to about 55° C., extended to the full original length (20 cm) and cooled again to room temperature. The permanent shape can be recovered by heating the straight strand to about 55° C. At this temperature, the strand spontaneously retracted to the permanent (curly) shape. In order to produce the permanent shape (e.g., straight) again, the curled strand was again heated to 55° C., extended to its full length (20 cm) and cooled to room temperature.

Examples with Cross-Linkable Macromers

The following hair treatment compositions were prepared using the macromers listed below which can be cross-linked into shape memory polymers. The production of the macromers was carried out as described in WO 99/42147.

M1 PEG (4k)-DMA, Polyethyleneglycol having a molecular weight of about 4,000, twice esterified with methacrylic acid

M2 PEG (8k)-DMA, Polyethyleneglycol having a molecular weight of about 8,000, twice esterified with methacrylic acid

M3 PEG (10k)-DMA, Polyethyleneglycol having a molecular weight of about 10,000, twice esterified with methacrylic acid

M4 PLG (7k)-DMA, Poly(L-lactide-co-glycolide)-dimethacrylate having a molecular weight of about 7,000

M5 PCI (10k)-DMA, Poly(ε-caprolactone)-dimethacrylate having a molecular weight of about 10,000

The application of the following hair treatment compositions was carried out as described in Example 1, with similar results.

### Example 13

### Non-Aerosol Spray Lotion

| | | |
|---|---|---|
| 5.0 | g | Macromer Ml |
| 0.5 | g | PVP/VA copolymer |
| 0.5 | g | Luviquat ® FC 550³⁾ |
| 0.5 | g | Tetraethoxylauryl ether |
| 1 | g | Dimethylpolysiloxane (Wacker-Belsil ® DM 500) |
| 0.5 | g | Polysorbate 40 |
| ad 100 | g | Ethanol |

³⁾40% solution of methyl vinyl imidazolium chloride/vinyl pyrrolidone copolymer (polyquaternary-16) in water

The lotion is sprayed directly onto the dry or clamp hair with a commercially available spray pump.

### Example 14

### Liquid-Fixer with Memory Effect

| | | |
|---|---|---|
| 6.0 | g | Macromer M2 |
| 5.0 | g | Cyclo-octamethyltetrasiloxane |
| 5.0 | g | Vinylpyrrolidone/vinyl acetate copolymer |
| 2.0 | g | Dihydroxy-polydimethylsiloxane, 12% in cyclo |
| | | dimethyl siloxane (Abil ® OSW 12 from the |
| | | Goldschmidt Company/Germany) |
| ad 100 | g | Pentane |

### Example 15

### Hair Wax with Memory Effect

| | | |
|---|---|---|
| 7 | g | Macromer M3 |
| 6 | g | Ethanol |
| 4 | g | Cetyl alcohol |
| 4 | g | Petrolatum |
| 3 | g | Ceresin |
| 3 | g | Carnauba wax |
| 2 | g | Ceteareth-25 |
| 2 | g | Polyvinylpyrrolidone |
| 1.5 | g | Mineral oil |
| 1.5 | g | Dicapryl maleate |
| 0.5 | g | Carbomer |
| 0.4 | g | Aminomethylpropanol |
| ad 100 | g | Water |

### Example 16

### Hair Fluid with Memory Effect

| 3 | g | Macromer M4 |
|---|---|---|
| 0.6 | g | Behenyltrimethyl ammonium chloride |
| 0.5 | g | Sunflower oil |
| 0.5 | g | Dimethylpolysiloxane (Wacker-Belsil ® DM 1000) |
| 0.5 | g | Abil Quat ® 3272 (Quaternium-80) |
| 0.5 | g | Guar hydroxypropyltrimonium chloride |
| 0.5 | g | Panthenol |
| 0.4 | g | Cetearyl alcohol |
| 0.2 | g | Beeswax |
| 00.1 | g | Cetyl alcohol |
| 0.1 | g | PEG-20 stearate |
| q.s. | | Preservative, aromatic substances |
| ad 100 | g | Water |

### Example 17

### Aerosol Foam with Memory Effect

| | | |
|---|---|---|
| 4 | g | Macromer M5 |
| 1 | g | Dow Corning DC 1401 Fluid |
| | | (dimethiconol/cyclomethicone 13/87) |
| 1 | g | Glucose |
| 0.6 | g | Polyvinylpyrrolidone |
| 0.8 | g | Aldi Quat ® 3272 (Quaternium-80) |
| 0.2 | g | Cetyl trimethyl ammonium chloride |
| 0.2 | g | Polyquaternium-11 |
| 0.2 | g | Silica |
| 0.1 | g | Laureth-2 |
| ad 100 | g | Water |

The composition, together with propane/butane (5 percent by weight), is filled into a pressure-proof aerosol package having a foam head.

In Examples 14 to 17, the particular macromer used can be completely or partially replaced with one of the other macromers M1 to M5 with similar results.

Examples with Thermoplastic Shape Memory Polymers

Each of the shape memory polymers used in the following cosmetic compositions is prepared from two different macrodiols and trimethylhexan-1,6-diisocyanate in a manner similar to that used in Example 1 of WO 99/42147.

| Macrodiol | P1 | P2 | P3 | P4 |
|---|---|---|---|---|
| PDX 1500 | 48 | | | |
| [% by weight] | | | | |
| PLGA 2000 | 52 | | | |
| [% by weight] | | | | |
| PCL 4000 | | 60 | | |
| [% by weight] | | | | |
| PCL 10000 | | | 50 | 60 |
| [% by weight] | | | | |
| PDL 2000 | | 40 | | |
| [% by weight] | | | | |
| PDL 3000 | | | 50 | 40 |
| [% by weight] | | | | |
| M_{w} copolymer [g/mol] | 79,100 | 150,000 | 130,000 | 115,000 |
| Tₜᵣₐₙₛ [° C.] | 34 | 48 | 55 | 55 |
| T'ₜᵣₐₙₛ [° C.] | 85 | 89 | 87 | 87 |

| Macrodiol | P5 | P6 | P7 | P8 |
|---|---|---|---|---|
| PCL 10000 | 78 | 59 | 40 | 20 |
| [% by weight] | | | | |
| PDL 10000 | 22 | 41 | 60 | 80 |
| [% by weight] | | | | |
| M_{w} copolymer [g/mol] | 208,000 | 357,000 | 282,000 | 300,100 |
| Tₜᵣₐₙₛ [° C.] | 55 | 54 | 55 | 54 |
| T'ₜᵣₐₙₛ [° C.] | 91 | 91 | 93 | 94 |

The abbreviations of the macrodiols have the following meaning:

### PDX: Poly(para-dioxanone)

### PLGA: Poly(L-lactide-co-glycolide)

PCL: Poly(ε-caprolactone)

### PDL: Poly(pentadecalactone)

The numbers presented in connection with the macrodiols represent the approximate molecular weight of the macrodiols (±1100).

The following hair treatment compositions are used as follows:

A sufficient amount, depending on the length of the hair, of the composition is applied to the hair. The hair is brought into the desired shape, for example by using curlers onto which the hair is wound, or by stretching the hair, followed by drying. Subsequently the hair is heated to about 70° C. After cooling to room temperature (about 25° C.) the curlers are removed. To impress a second hairstyle (temporary shape), the first hairstyle is heated to about 55° C., brought into a second hairstyle and cooled to room temperature. When reheated to about 55° C., the first hairstyle is spontaneously recovered.

### Example 18

### Hair Cream with Shape Memory Effect

| | | |
|---|---|---|
| 5 | g | Polymer P1 |
| 0.5 | g | Cyclooctamethyltetrasiloxane |
| 2 | g | Acrylate/alkyl acrylate copolymer (Pemulen ® TR1) |
| 1 | g | Abil ® OSW 13 (dimethiconol, 13% in cyclomethicone) |
| 5 | g | Water |
| ad 100 | g | Isopropanol |

### Example 19

### Aerosol Foam with Memory Effect

| | | |
|---|---|---|
| 6 | g | Polymer P2 |
| 1 | g | Dow Corning DC 1401 Fluid |
| | | (dimethiconol/cyclomethicone 13/87) |
| 0.2 | g | Polyvinylpyrrolidone |
| 0.8 | g | Abil Quat ® 3272 (Quaternium-80) |
| 0.3 | g | Cetyl trimethyl ammonium chloride |
| 0.2 | g | Polyquaternium-11 |
| 0.2 | g | Panthenol |
| 0.1 | g | Jojoba oil, orange peel wax, apple peel wax |
| ad 100 | g | Water |

The composition, together with propane/butane (5 percent by weight), is filled into a pressure-proof aerosol package having a foam head.

### Example 20

### Liquid Colouring Fixative with Memory Effect

| | | |
|---|---|---|
| 4.0 | g | Polymer P7 |
| 2.0 | g | Cyclooctamethyltetrasiloxane |
| 0.5 | g | Dimethiconol (Silicone Fluid F 212 from the |
| | | Wacker Company/Germany) |
| 0.07 | g | 1-amino-4-(2;3-dihydroxypropyl)amino-5-chloro- |
| | | 2-nitrobenzol |
| 0.05 | g | Basic Brown 17 (C.I. 12251) |
| 0.0023 | g | Basic Violet 14 (C.I. 42595) |
| 0.01 | g | Basic Blue 7 (CI. 42595) |
| 20.0 | g | Pentane |
| ad 100 | g | Propanol |

### Example 21

### Hairspray with Memory Effect

| | | |
|---|---|---|
| 5.0 | g | Polymer P8 |
| 0.5 | g | Cyclooctamethyltetrasiloxane |
| 0.5 | g | Dimethiconol (Silicone Fluid F 212 from the |
| | | Wacker Company/Germany) |
| 5.0 | g | Vinylpyrrolidone/vinyl acetate copolymer |
| 35 | g | Propane/butane |
| ad 100 | g | Pentane |

### Example 22

### Hair Wax with Memory Effect

| | | |
|---|---|---|
| 8 | g | Polymer P3 |
| 5 | g | Glyceryl stearate |
| 4 | g | Cocamide MEA |
| 3 | g | Ceresin |
| 3 | g | Isopropyl myristate |
| 3 | g | Triceteareth-4 phosphate |
| 2 | g | Laureth-4 |
| 1 | g | Ceteareth-25 |
| 0.5 | g | Cetyl trimethyl ammonium chloride |
| 0.5 | g | Polyvinylpyrrolidone/vinyl acetate copolymer |
| ad 100 | g | Water |

In Examples 18 to 22, the particular shape memory polymer used can be completely or partially replaced with one of the other polymers P1 to P8 with similar results.

1. Method for hair treatment, wherein
an active ingredient composition is applied to the hair,
wherein the active ingredient composition
(A) contains at least a first active ingredient or first active ingredient complex, selected from or formed of substances which, either alone or in combination with other substances, can provide a shape memory effect after being applied to the hair and after the performance of the treatment of the hair described in the following, and wherein the active ingredient composition
(B) contains at least a second active ingredient, which is selected from the hydrophobic substances with a water solubility of ≦5 percent by weight at 20° C.;
the hair is arranged in a given shape (permanent memory shape) before, simultaneously with or after the application of the active ingredient composition and
the memory shape is subsequently fixed by inducing a chemical or physical modification of the applied active ingredients;
wherein after a wanted or unwanted deformation of the memory shape, the original memory shape can essentially be restored by means of a physical stimulation.

2. Method according to claim 1, characterised in that at least two substances are contained in the active ingredient composition, said substances having no or only weak shape memory characteristics individually, and said substances giving a synergistically increased shape memory effect to the hair when applied together according to the method according to claim 1.

3. Method according to claim 1, wherein
the first active ingredient is a cross-linkable macromer, which forms a shape memory polymer after cross-linking, wherein the macromer
a) contains cross-linkable regions that are cross-linkable through chemical bonds and
b) contains thermoplastic regions that are not chemically cross-linkable,
the memory shape is fixed by chemically cross-linking the macromer while forming the shape memory polymer,
and
wherein the shape memory polymer has at least one transition temperature Tₜᵣₐₙₛ.

4. Method for hair treatment, wherein
a hairstyle (permanent shape) programmed by means of a method according to claim 3 is heated to a temperature higher than Tₜᵣₐₙₛ,
the hair is brought into a second (temporary) shape and
the second shape is fixed by means of cooling to a temperature below Tₜᵣₐₙₛ.

5. Method according to claim 3 or 4, characterised in that the cross-linkable macromer is selected from compounds with the general formula
A1-(X)ₙ-A2
wherein A1 and A2 represent reactive, chemically cross-linkable groups and —(X)ₙ— represents a divalent, thermoplastic polymer or oligomer segment.

6. Method according to claim 5, characterised in that the cross-linkable macromer is selected from polyesters, oligoesters, polyalkylene glycols, oligoalkylene glycols, polyalkylene carbonates and oligoalkylene carbonates, substituted with at least two acrylate groups or methacrylate groups.

7. Method according to claim 6, characterised in that the cross-linkable macromer is selected from poly(ε-caprolactone)-dimethacrylate, poly(DL-lactide)-dimethacrylate, poly(L-lactide-co-glycolide)-dimethacrylate, poly(ethylene glycol)-dimethacrylate, poly(propylene glycol)-dimethacrylate, PEG-block-PPG-block-PEG-dimethacrylate, poly(ethylene adipate)-dimethacrylate, hexamethylene carbonate dimethacrylate.

8. Method according to one of the preceding claims, characterised in that the composition additionally contains a macromer with only one terminal or side chemically reactive group.

9. Method according to claim 1, wherein
the first active ingredient is a shape memory polymer that
a) has at least one hard segment that can be cross-linked by means of physical interaction, said hard segment having a first transition temperature T′ₜᵣₐₙₛ, which is above room temperature, and
b) has at least one soft segment with a second transition temperature Tₜᵣₐₙₛ, which is lower than T′ₜᵣₐₙₛ and
the memory shape is fixed by physical cross-linking of the shape memory polymers.

10. Method according to claim 9, characterised in that the shaping of the hair occurs under heating to a temperature of at least T′ₜᵣₐₙₛ, and that the subsequent fixing of the shape of the hair is achieved by means of cooling to a temperature below T′ₜᵣₐₙₛ.

11. Method for hair treatment, wherein
a hairstyle (permanent shape) programmed by a method according to claim 9 or 10 is heated to a temperature between T′ₜᵣₐₙₛ and Tₜᵣₐₙₛ,
the hair is brought into a second (temporary) shape and
the second shape is fixed by means of cooling to a temperature below Tₜᵣₐₙₛ.

12. Method for reprogramming a hairstyle (permanent shape) previously programmed by means of a method according to claim 9 into a new permanent shape, wherein
the hairstyle is heated to a temperature above T′ₜᵣₐₙₛ
is brought into a new shape and
the new shape is subsequently fixed by means of cooling to a temperature below T′ₜᵣₐₙₛ.

13. Method according to one of the claims 9 to 12, characterised in that the shape memory polymer has a degree of crystallinity of from 3 to 80% and that the ratio of the moduli of elasticity below and above Tₜᵣₐₙₛ is at least 20.

14. Method according to one of the claims 9 to 13, characterised in that the shape memory polymer is a copolyester urethane.

15. Method according to claim 14, characterised in that the shape memory polymer is the reaction product of (a) two different macrodiols, selected from α, ω-dihydroxy polyesters, α, ω-dihydroxy oligoesters, α, ω-dihydroxy polylactones and α, ω-dihydroxy oligolactones, and (b) at least one diisocyanate.

16. Method for the recovery of a hairstyle (permanent shape) previously programmed by means of a method according to claim 1, 3, 9 or 12, wherein a hairstyle in a temporary shape according to claim 4 or claim 11 or a hairstyle deformed by means of cold forming is heated to a temperature above Tₜᵣₐₙₛ.

17. Method according to one of the preceding claims, characterised in that the hydrophobic, second active ingredient is selected from dimerdiol, dimerdiol oligoethers, oligoester diols, dimer acid, PMS divinyl ether, hydrophobic polymers, hydrophobic tensides with HLB levels≦7, silicone compounds, organic oils, fats, waxes, glyceryl esters, fatty acid esters, fatty alcohol esters.

18. Method according to claim 17, characterised in that the hydrophobic polymer is selected from polymers that contain at least one type of monomer, which is selected from acrylic acid alkyl esters and methacrylic acid alkyl esters, wherein the alkyl groups have at least 6 C atoms.

19. Method according to claim 17, characterised in that the silicone compounds are selected from dimethylpolysiloxanes and dimethylsiloxane/alkylmethylsiloxane copolymers, wherein the alkyl groups have at least 6 C atoms.

20. Method according to one of the preceding claims, characterised in that the active ingredient composition contains the first active ingredient in an amount of 0.01 to 25 percent by weight and the second active ingredient in an amount of 0.01 to 25 percent by weight.

21. Method according to one of the preceding claims, characterised in that the active ingredient composition is present as a solution and contains water and at least one organic solvent.

22. Method according to claim 21, characterised in that the organic solvents are selected from ethanol, isopropanol, n-propanol and acetone.

23. Hair cosmetic composition containing an active ingredient complex in a suitable cosmetic foundation, wherein the active ingredient complex
(A) contains at least a first active ingredient which is selected from substances which, alone or in combination with other substances, can provide a shape memory effect to the hair after being applied to the hair and after the performance of a method according to one of the claims 1 to 20, and
(B) contains at least a second active ingredient, which is selected from hydrophobic substances with a water solubility of ≦5 percent by weight at 20° C.

24. Composition according to claim 23, characterised in that the first active ingredient (A) is either a macromer that can be cross-linked into a shape memory polymer, wherein the cross-linked shape memory polymer has at least one transition temperature Tₜᵣₐₙₛ and wherein the macromer
a) contains cross-linkable regions that are cross-linkable through chemical bonds and
b) contains thermoplastic regions that are not chemically cross-linkable;
and/or that the first active ingredient is a shape memory polymer that
a) has at least one hard segment that can be cross-linked by means of physical interaction, said hard segment having a first transition temperature T′ₜᵣₐₙₛ, which is above room temperature, and
b) has at least one soft segment with a second transition temperature Tₜᵣₐₙₛ, which is less than T′ₜᵣₐₙₛ;
and the second active ingredient (B) is selected from dimerdiols, dimerdiol oligoethers, oligoester diols, dimer fatty acids, PMS-divinyl ethers, hydrophobic polymers, hydrophobic tensides with HLB levels≦7, silicone compounds, organic oils, fats and waxes; wherein the weight ratio of (A):(B) is from 1:1 to 2.5:1.

25. Composition according to claim 24, characterised in that the hydrophobic polymer is selected from polymers that contain at least one type of monomer, which is selected from acrylic acid alkyl esters and methacrylic acid alkyl esters, wherein the alkyl groups have at least 6 C atoms.

26. Composition according to claim 24, characterised in that the silicone compounds are selected from dimethylpolysiloxanes and dimethylsiloxane/alkylmethylsiloxane copolymers, wherein the alkyl groups have at least 6 C atoms.

27. Composition according to one of the claims 23 to 26, characterised in that the shape memory polymer is contained at 0.01 to 25% percent by weight and the hydrophobic active ingredient in an amount of from 0.01 to 25 percent by weight.

28. Composition according to one of the claims 23 to 27, characterised in that at least two substances are contained, said substances having no or only weak shape memory characteristics individually and said substances giving a synergistically increased shape memory effect to the hair when applied together according to the method according to claim 1.

29. Cosmetic substance containing a composition according to one of the claims 23 to 28, characterised in that it is present in the form of a lotion, a spray lotion, a cream, a gel, a foam-gel, an aerosol spray, a non-aerosol spray, an aerosol foam, a non-aerosol foam, an o/w or a w/o emulsion, a micro-emulsion or a hair wax.

30. Substance according to claim 29, characterised in that an additional 0.01 to 25 percent by weight of at least one active ingredient is contained, said active ingredient being selected from hair-care substances, hair-fixing substances and hair-colouring substances.
